(19) United States
(12) Patent Application Publication
(10) Pub. No.: US 2024/0189485 A1
(43) Pub. Date: Jun. 13, 2024

(54) NOVEL RADIOPAQUE MEDICAL HYDROGELS AND PRECURSORS THEREOF

(71) Applicant: Boston Scientific Scimed, Inc., Maple Grove, MN (US)

(72) Inventors: Cristian Parisi, Boston, MA (US); Yen-Hao Hsu, JR., Shrewsbury, MA (US); Joseph Thomas Delaney, JR., Minneapolis, MN (US)

(73) Assignee: Boston Scientific Scimed, Inc., Maple Grove, MN (US)

(21) Appl. No.: 18/516,271

(22) Filed: Nov. 21, 2023

Related U.S. Application Data
(60) Provisional application No. 63/428,010, filed on Nov. 25, 2022.

(51) Int. Cl. A61L 27/50; A61L 27/16

(52) U.S. Cl. CPC A61L 27/50; A61L 27/16; A61L 2300/202

(57) ABSTRACT

The disclosure pertains to radiopaque, unsaturated multi-arm polymers that comprise a plurality of hydrophilic polymer arms having end moieties comprising one or more radiopaque halogen groups and one or more unsaturated groups, the end moieties linked to the hydrophilic polymer arms though an ester or an amide linkage. In some embodiments, the disclosure pertains to systems for forming hydrogels, which systems comprise (a) a polyamino compound and (b) a radiopaque, unsaturated multi-arm polymer that comprises a plurality of hydrophilic polymer arms having end moieties comprising one or more radiopaque halogen groups and one or more unsaturated groups, the end moieties linked to the hydrophilic polymer arms though an ester or an amide linkage, wherein the one or more unsaturated groups are reactive with the amino groups of the polyamino compound. Other embodiments pertain to methods of treatment using such systems and to hydrogels formed from such systems.

### CROSS-REFERENCE TO RELATED APPLICATIONS

This application claims the benefit of U.S. Provisional Patent Application Ser. No. 63/428,010 filed on Nov. 25, 2022, the disclosure of which is incorporated herein by reference.

### FIELD

The present disclosure relates to radiopaque crosslinked hydrogels, to methods of making and using radiopaque crosslinked hydrogels and to precursors thereof, among other aspects. The radiopaque crosslinked hydrogels of the present disclosure are useful, for example, in various medical applications.

### BACKGROUND

In vivo crosslinked hydrogels based on star-poly(ethylene glycol) (star-PEG) polymers functionalized with reactive ester end groups which react with lysine trimer (Lys-Lys-Lys) as a crosslinker to rapidly form crosslinked hydrogels, such as SpaceOAR®, have become clinically significant materials as adjuvants in radiotherapies. See “Augmenix Announces Positive Three-year SpaceOAR® Clinical Trial Results,” Imaging Technology News, Oct. 27, 2016.

Hydrogels in which some of the star-PEG branches have been functionalized with 2,3,5-triiiodobenzamide (TIB) groups replacing part of ester end groups, such as SpaceOAR® Vue, have also been developed to provide enhanced radiocontrast properties. See “Augmenix Receives FDA Clearance to Market its TraceIT® Tissue Marker,” Business Wire Jan. 28, 2013. TraceIT® hydrogel remains stable and visible in tissue for a minimum of three months, long enough for radiotherapy, after which it is absorbed and cleared from the body.

While the above approach is effectual, some issues arise as a result of incorporation of the functional group, TIB. First, in order to functionalize TIB on 8-arm PEG, the binding site from succinimidyl glutarate (SG) must be sacrificed for each functionalized arm. Moreover, the entire functionalization process involves multiple steps, typically five steps, from commercially available hydroxyl-terminated 8-arm PEG to its functionalized form with two different end groups (TIB and SG groups). This complex process of synthesizing the 8-arm PEG results in a significant increase of the product cost, decreased hydrogel persistence, and difficulties in product quality control. Furthermore, each added TIB group occupies one arm of the star-PEG, reducing capacity and efficiency of the crosslinking reaction. Lastly, SG groups can start their degradation in acidic pH environments, which could potentially cause longer gel times and faster dissipation of crosslinked hydrogels in vivo.

### SUMMARY

The present disclosure is directed to an alternative approach to that described above, in which a radiopaque precursor compound having one or more radiopaque halogen groups, one or more carboxy groups and one or more hydroxyl groups are used to incorporate radiopaque halogen atoms on multi-arm polymers, followed by a further reaction with a reagent suitable for the creation of unsaturated functional groups as active end groups. This two-step functionalization strategy not only reduces the synthetic complexity of incorporating iodine atoms on 8-arm PEG as described above, but also provides a refined crosslinking option based on the unsaturated end groups via Michael addition, allowing the use of reactive succinimidyl glutarate groups on the multi-arm polymer to be avoided.

In various embodiments, the present disclosure pertains to radiopaque, unsaturated multi-arm polymers that comprise a plurality of hydrophilic polymer arms having end moieties comprising one or more radiopaque halogen groups and one or more unsaturated groups, the end moieties linked to the hydrophilic polymer arms though an ester or an amide linkage.

In some embodiments, which can be used in conjunction with any of the preceding embodiments, the hydrophilic polymer arms are selected from polyether arms, poly(N-vinyl pyrrolidone) arms, polyoxazoline arms, poly(vinyl alcohol) arms, poly(allyl alcohol) arms, polyhydroxyethyl acrylate arms, polyhydroxyethyl methacrylate arms, PNIPAAM arms, and polysaccharide arms.

In some embodiments, which can be used in conjunction with any of the preceding embodiments, hydrophilic polymer arms originate from a core region comprising a polyol residue or a core region comprising a silsesquioxane.

In some embodiments, which can be used in conjunction with any of the preceding embodiments, the radiopaque halogen groups are iodine groups and the one or more unsaturated groups are selected from acrylate ester groups and propiolate ester groups.

In some embodiments, which can be used in conjunction with any of the preceding embodiments, the end moieties comprise a monocyclic or multicyclic aromatic moiety that is substituted with (a) one or more iodine groups and (b) one or more acrylate groups and/or one or more C₄-C₁₆-alkyl acrylate groups.

In some embodiments, which can be used in conjunction with any of the preceding embodiments, the end moieties comprise a benzene ring that is substituted with one or more iodine groups and one or more acrylate groups or propiolate groups.

In various embodiments, the present disclosure pertains to systems for forming a hydrogel, which systems comprise (a) a polyamino compound and (b) a radiopaque, unsaturated multi-arm polymer in accordance with any of the preceding embodiments, wherein the one or more unsaturated groups of the radiopaque, unsaturated multi-arm polymer are reactive with the amino groups of the polyamino compound.

In some embodiments, which can be used in conjunction with the

preceding embodiments, the polyamino compound comprises a plurality of —(CH₂)ₓ—NH2 groups where x is 0, 1, 2 3, 4, 5 or 6. In some of these embodiments, the plurality of —(CH₂)ₓ—NH₂ groups are disposed along a polymeric moiety.

In some embodiments, which can be used in conjunction with any of the preceding embodiments, the polyamino compound comprises two or more amino acid residues selected from residues of lysine, ornithine, and combinations thereof.

In some embodiments, which can be used in conjunction with any of the preceding embodiments, the system comprises a first precursor composition that comprises the polyamino compound, a second precursor composition that comprises the radiopaque, unsaturated multi-arm polymer, and an optional accelerant composition.

In some embodiments, which can be used in conjunction with any of the preceding embodiments, the system comprises a first precursor composition that comprises the polyamino compound in a first syringe barrel, a second precursor composition that comprises the radiopaque, unsaturated multi-arm polymer in a vial, and an accelerant composition in a second syringe barrel.

In some embodiments, which can be used in conjunction with any of the preceding embodiments, the system further comprises a delivery device.

In various embodiments, the present disclosure pertains to medical hydrogels that are formed by crosslinking the polyamino compound of any of the preceding embodiments and the radiopaque, unsaturated multi-arm polymer of any of the preceding embodiments under conditions such that a medical hydrogel is formed. In some embodiments, the medical hydrogel is a medical implant.

In various embodiments, the present disclosure pertains to methods of treatment comprising administering to a subject a mixture that comprises the polyamino compound of any of the preceding embodiments and the radiopaque, unsaturated multi-arm polymer of any of the preceding embodiments under conditions such that the polyamino compound and the radiopaque, unsaturated multi-arm polymer cross-link after administration.

It should be noted that, while radiopaque halogen atoms have been described in the present disclosure, other radiopaque atoms can be substituted for the radiopaque halogen atoms.

Potential benefits associated with the present disclosure include one or more of the following: radiocontrast is maintained, complexity and cost of the manufacturing process is reduced, melting point of the solid components of the hydrogel can be maintained above 40° C. (improving storage and handling), homogeneity of the final hydrogel is improved, in vivo persistence is obtained, side products generation during crosslinking are eliminated, and cure kinetics are maintained.

The above and other aspects, embodiments, features and benefits of the present disclosure will be readily apparent from the following detailed description.

### BRIEF DESCRIPTION OF THE DRAWINGS

FIGS. 1A and 1B schematically illustrate a method whereby polymers are produced, according to two aspects of the present disclosure.

FIG. 2 schematically illustrates a method whereby a polymer, which comprises a core region and a plurality of hydrophilic polymer arms having acrylate end groups is crosslinked with a polyamino compound, according to an aspect of the present disclosure.

### DETAILED DESCRIPTION

In some aspects of the present disclosure, radiopaque crosslinked hydrogels are provided, which are crosslinked reaction products of (a) one or more radiopaque, unsaturated multi-arm polymers and (b) one or more polyamino compounds. Unless indicated otherwise, as used herein the prefix “poly” means two or more. Particular radiopaque, unsaturated multi-arm polymers and particular polyamino compounds are described below.

Radiopaque, unsaturated multi-arm polymers having arms that comprise one or more end moieties having one or more radiopaque halogen groups and one or more unsaturated groups (e.g., acrylate ester groups, propiolate ester groups, etc.) can be formed from precursor multi-arm polymers having arms that comprise one or more hydroxyl end groups or one or more amino end groups.

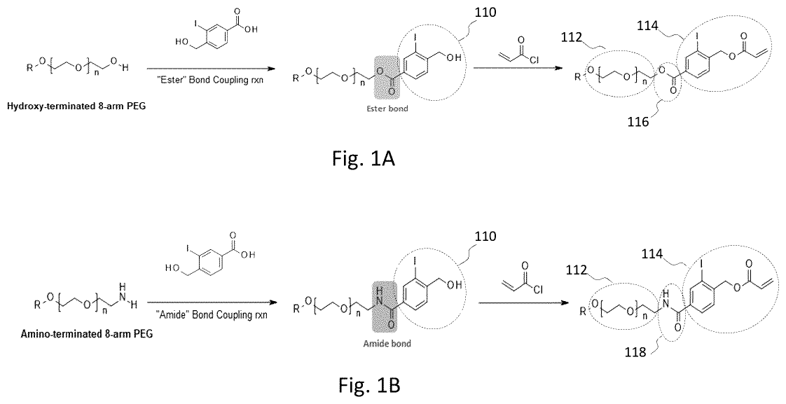

In one exemplary embodiment shown in FIG. 1A, hydroxyl groups of a precursor multi-arm polymer, which comprises a core region and a plurality of polymer arms (e.g., two, three, four, five, six, seven, eight, nine, ten, eleven, twelve or more polymer arms) having terminal hydroxyl groups, are reacted in an esterification reaction of the hydroxyl groups with carboxy groups of a radiopaque precursor compound having one or more radiopaque halogen groups, one or more carboxy groups and one or more hydroxyl groups. More specifically, a commercially available hydroxy-terminated 8-arm PEG having a core region that comprises a polyol residue R and eight hydroxyl-terminated polyethylene oxide arms, where n ranges from 30 to 140, and 4-(hydroxymethyl)-3-iodobenzoic acid (CAS #1824579-84-1) are coupled by forming an ester bond linkage. Such an ester coupling reaction may be performed using a suitable coupling reagent, for instance, a carbodiimide coupling reagent such as dicyclohexylcarbodiimide (DCC) or diisopropylcarbodiimide (DIC). This first reaction step forms an intermediate multi-arm polymer, which comprises a core region R and a plurality of polymer arms having end moieties 110 that comprise one or more radiopaque halogen groups and one or more hydroxyl groups. In a second step, reactive unsaturated end groups, specifically, acrylate end groups, are formed by reacting hydroxyl groups of the end moieties 110 of the intermediate multi-arm polymer with acryloyl chloride, resulting in a radiopaque, unsaturated multi-arm polymer that comprises a core region R and a plurality of polymer arms 112 having end moieties 114 that comprise one or more radiopaque halogen groups and one or more acrylate groups that are linked to the polymer arms 112 by an ester group 116.

In FIG. 1B, on the other hand, a radiopaque, unsaturated multi-arm polymer is formed that comprises a core region R and a plurality of polymer arms 112 having end moieties 114 that comprise one or more radiopaque halogen groups and one or more acrylate groups that are linked to the polymer arms 112 by an amide group 118. Amide groups are known to be more resistant to hydrolysis and bond degradation that ester groups, which degrade more readily in vivo than amide groups.

In a first step shown in FIG. 1B, amino groups of a precursor multi-arm polymer, which comprises a core region and a plurality of polymer arms having amino terminal groups, are reacted in an amidation reaction with carboxy groups of a radiopaque precursor compound having one or more radiopaque halogen groups, one or more carboxy groups and one or more hydroxyl groups. More specifically, amino-terminated 8-arm PEG having a core region that comprises a polyol residue R and eight amino-terminated polyethylene oxide arms where n ranges from 30 to 140 may be coupled to 4-(hydroxymethyl)-3-iodobenzoic acid (CAS #1824579-84-1) by forming an amide bond linkage. Such an amide coupling reaction may be performed using a suitable coupling reagent, for instance, a carbodiimide coupling reagent such as 1-ethyl-3-(3-dimethylaminopropyl)carbodiimide hydrochloride (EDC·HCl). This first reaction step forms an intermediate multi-arm polymer, which comprises a core region and a plurality of polymer arms having end moieties 110 that comprise one or more radiopaque halogen groups and one or more hydroxyl groups that are linked to the polymer arms by an amide group. In a second step, reactive acrylate end groups are formed by reacting hydroxyl groups of the end moieties 110 of the intermediate multi-arm polymer with acryloyl chloride, resulting in a multi-arm polymer that comprises a core region R and a plurality of polymer arms having end moieties 114 that comprise one or more radiopaque halogen groups and one or more acrylate groups.

Each of the above reaction schemes shown in FIGS. 1A and 1B require a radiopaque precursor compound having one or more radiopaque halogen groups, one or more carboxy groups and one or more hydroxyl groups. In various embodiments, this compound may comprise a monocyclic or multicyclic aromatic structure, such as a benzene or naphthalene structure, that is substituted with (a) one or more iodine groups, (b) one or more carboxyl groups or carboxyl-containing groups such as C₂-C₈-carboxyalkyl groups (e.g., C₂-C₅-monocarboxyalkyl groups, C₃-C₆-dicarboxyalkyl groups, C₄-C₇-tricarboxyalkyl groups, C₅-C₈-tetracarboxyalkyl groups, etc.), among others, which carboxyalkyl groups may be linked to the monocyclic or multicyclic aromatic structures directly or through any suitable linking moiety, which may be selected, for example, from amide groups, amine groups, ether groups, ester groups, or carbonate groups, among others, and (c) one or more hydroxyl groups or hydroxyl-containing groups such as C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which hydroxyalkyl groups may be linked to the monocyclic or multicyclic aromatic structures directly or through any suitable linking moiety, which may be selected, for example, from amide groups, amine groups, ether groups, ester groups, or carbonate groups, among others. Specific examples of such compounds include the following, among many others: hydroxymethyl iodobenzoic acid,

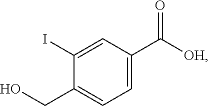 [SMILES: O=C(O)c1ccc(CO)c(I)c1]

hydroxymethyl diiodobenzoic acid,

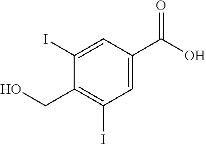 [SMILES: O=C(O)c1cc(I)c(CO)c(I)c1]

and tetraiodoterephthalic acid,

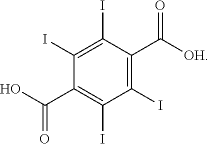 [SMILES: O=C(O)c1c(I)c(I)c(C(=O)O)c(I)c1I]

Although iodine groups are described, other radiopaque halogen groups including bromine may be employed.

Each of the above reaction schemes also requires a precursor multi-arm polymer having a plurality of polymer arms (e.g., having two, three, four, five, six, seven, eight, nine, ten, eleven, twelve or more arms), wherein two or more polymer arms of the precursor multi-arm polymer each comprises one or more hydroxyl end groups or one or more amino end groups.

In various embodiments, the polymer arms are hydrophilic polymer arms. Such hydrophilic polymer arms may be composed of any of a variety of synthetic, natural, or hybrid synthetic-natural polymer arms. These may be selected, for example, from the following polymer arms: polyether arms including poly(alkylene oxide) arms such as poly(ethylene oxide) (PEO) (also referred to as polyethylene glycol or PEG) arms, poly(propylene oxide) arms, poly(ethylene oxide-co-propylene oxide) arms, poly(N-vinyl pyrrolidone) arms, polyoxazoline arms including poly(2-alkyl-2-oxazoline) arms such as poly(2-methyl-2-oxazoline) arms, poly(2-ethyl-2-oxazoline) arms and poly(2-propyl-2-oxazoline) arms, poly(vinyl alcohol) arms, poly(allyl alcohol) arms, polyhydroxyethyl acrylate arms, polyhydroxyethyl methacrylate arms, PNIPAAM arms, or polysaccharide arms. Polymer arms for use in the multi-arm polymers of the present disclosure typically contain between 10 and 1000 monomer units.

In various embodiments, the polymer arms extend from a core region. In certain of these embodiments, the core region comprises a residue of a polyol that is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, tripentaerythritol adonitol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl)alkanes, such as 1,1,1-tris(4-hydroxyphenyl)ethane, and 2,6-bis(hydroxyalkyl)cresols, among others. In certain beneficial embodiments, the core region comprises a residue of a polyol that contains two, three, four, five, six, seven, eight, nine, ten or more hydroxyl groups.

In certain of these embodiments, the core region comprises a silsesquioxane. A silsesquioxane is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers, -. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

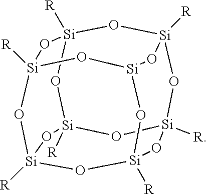

In the present disclosure, at least two R groups comprise polymer arms, and typically all R groups comprise polymer arms.

Each of the above reaction schemes produces an intermediate multi-arm polymer having a plurality of polymer arms (e.g., having two, three, four, five, six, seven, eight, nine, ten, eleven, twelve or more arms), wherein two or more polymer arms of the intermediate multi-arm polymer each comprise one or more end moieties that comprise one or more radiopaque halogen groups and one or more hydroxy groups, which end moieties are linked to the polymer arms by an ester linkage or an amide linkage. In various embodiments, this compound may comprise a monocyclic or multicyclic aromatic structure, such as a benzene or a naphthalene structure, that is substituted with (a) one or more iodine groups and (b) one or more hydroxyl groups or hydroxyl-containing groups such as C₁-C₄-hydroxyalkyl groups (e.g., C₁-C₄-monohydroxyalkyl groups, C₁-C₄-dihydroxyalkyl dihydroxyalkyl groups, C₁-C₄-trihydroxyalkyl groups, C₁-C₄-tetrahydroxyalkyl groups, etc.), among others, which hydroxyalkyl groups may be linked to the monocyclic or multicyclic aromatic structures directly or through any suitable linking moiety, which may be selected, for example, from amide groups, amine groups, ether groups, ester groups, or carbonate groups, among others. Specific examples of such end moieties include iodobenzoic add and diiodobenzoic add moieties, among others, Although iodine groups are described, other radiopaque halogen groups including bromine may be employed.

Each of the above reaction schemes also produces a radiopaque, unsaturated multi-arm polymer having a plurality of polymer arms (e.g., having two, three, four, five, six, seven, eight, nine, ten, eleven, twelve or more arms), wherein two or more polymer arms of the radiopaque, unsaturated multi-arm polymer each comprise one or more end moieties that comprise one or more radiopaque halogen groups and one or more unsaturated groups, which end moieties are linked to the polymer arms by an ester linkage or an amide linkage. Examples of polymer arms and core regions are described above. Examples of radiopaque halogen groups include iodine groups and bromine groups. Examples of unsaturated groups include unsaturated groups having double carbon-carbon bonds such as acrylate ester groups,

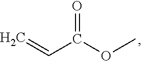

and unsaturated groups having triple carbon-carbon bonds such as propiolate ester groups,

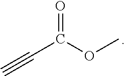

In various embodiments, such end moieties may comprise a monocyclic or multicyclic aromatic structure, such as a benzene or naphthalene moiety, that is substituted with (a) one or more iodine groups (b) one or more acrylate groups or acrylate containing groups such as C₄-C₁₆-alkyl acrylate groups (e.g., C₄-C₇-alkyl monoacrylate groups, C₇-C₁₀-alkyl diacrylate groups, C₁₀-C₁₃-alkyl triacrylate groups, etc.), among others, which alkyl acrylate groups may be linked to the monocyclic or multicyclic aromatic structures directly or through any suitable linking moiety. Although acrylate groups are described, other unsaturated groups including propiolate groups may be employed. Moreover, although iodine groups are described other radiopaque halogen groups including bromine may be employed.

As previously indicated, in some aspects, the present disclosure provides a radiopaque medical hydrogel that comprises a crosslinked reaction product of (a) a polyamino compound and (b) a radiopaque, unsaturated multi-arm polymer like that described above.

More particularly, in some aspects of the present disclosure, a radiopaque crosslinked hydrogel is provided that comprises a crosslinked reaction product of (a) a polyamino compound and (b) a radiopaque, unsaturated multi-arm polymer that comprises a plurality of polymer arms that have end moieties comprising one or more radiopaque halogen groups and one or more unsaturated groups, such as those described above, which unsaturated groups are reactive with the amino groups of the polyamino compound via Michael addition.

In general, polyamino compounds suitable for use in the present disclosure include, for example, small molecule polyamines (e.g., containing at least two amine groups, for instance, from 3 to 20 amine groups or more in certain embodiments), polymers having amine side groups, and branched polymers having amine end groups, including dendritic polymers having amine end groups. Polyamino compounds suitable for use in the present disclosure include those that comprises a plurality of —(CH₂)ₓ—NH₂ groups where x is 0, 1, 2, 3, 4, 5 or 6. Polyamino compounds suitable for use in the present disclosure include polyamino compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Particular examples of polyamino compounds which may be used as the multifunctional compound include ethylenetriamine, diethylene triamine, hexamethylenetriamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, and poly(allyl amine), among others among others.

As noted above, the amino groups of the polyamino compound react with the unsaturated groups of the radiopaque, unsaturated multi-arm polymer via Michael addition. In certain embodiments, reaction between the unsaturated groups and the amino groups is conducted at slightly basic pH (e.g., having a pH value ranging from 7.4 to 11) where the amino groups of the polyamino compound are deprotonated/neutrally charged and the Michael addition can occur spontaneously at body temperature.

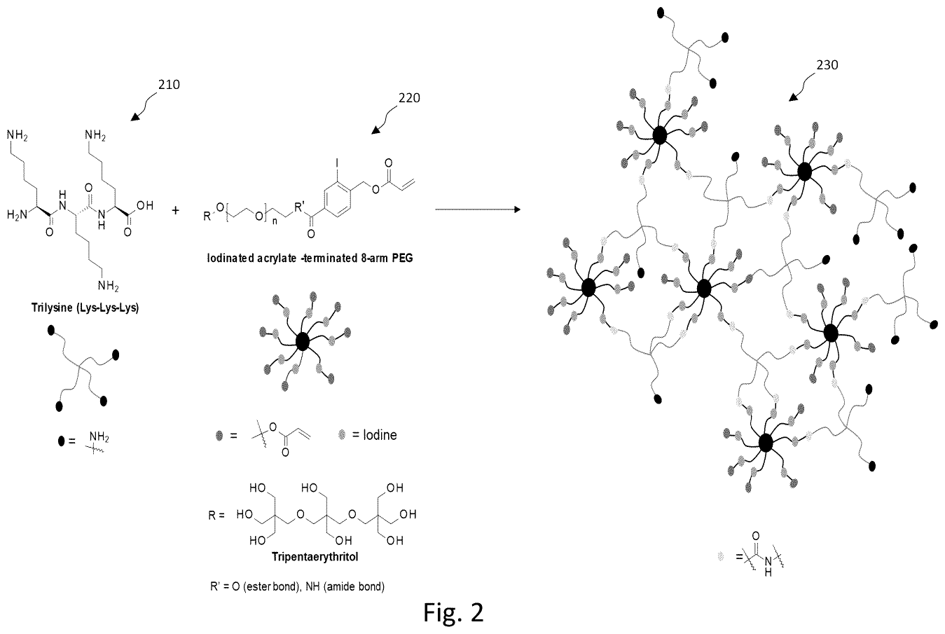

In an embodiment of the present disclosure, and with reference to FIG. 2, a polyamino compound, specifically, a trilysine peptide oligomer (Lys-Lys-Lys) 210 is combined with a radiopaque, unsaturated multi-arm polymer like that described above, for example, having a core R (e.g., a pentaerythritol residue core) and a plurality of (e.g. eight) PEG arms with end moieties comprising one or more radiopaque halogen groups (e.g., an iodine group) and one or more unsaturated groups (e.g., an acrylate group) 220, under conditions such that Michael addition occurs between the acrylate groups of the acrylate-terminated PEG 220 and the amino groups of the trilysine 210, thereby forming a crosslinked hydrogel 230. For example, the Michael addition can proceed in aqueous solution under slightly basic conditions (e.g., at a pH ranging from 7.4 to 11) in the presence of buffer solution such as phosphate-buffer saline (PBS) or a borax-related buffer solution, among others.

An advantage to this approach is that the relative number of radiopaque halogen groups and unsaturated groups can be varied (based on the radiopaque precursor compound selected for the synthesis), and the polyethylene oxide arms can be replaced by other hydrophilic polymer arms, for example, synthetic, natural, or hybrid synthetic-natural hydrophilic polymer arms such as those described above.

In various embodiments, the crosslinked hydrogels of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including medical devices, implants, and pharmaceutical compositions.

In some aspects of the present disclosure, a system is provided that is configured to dispense a polyamino compound and a radiopaque, unsaturated multi-arm polymer under conditions such that the polyamino compound and the polymer crosslink with one another. In certain embodiments, those conditions comprise an environment having a basic pH, for example, a pH ranging from about 9.0 to about 11, typically ranging from about 9.5 to about 10.5, and beneficially ranging from about 9.8 to about 10.2. Such systems can be used to form crosslinked medical hydrogels, either in vivo or ex vivo.

For example, in some aspects of the present disclosure, systems are provided that comprise (a) a first composition that comprises a polyamino compound in accordance with the present disclosure and (b) a second composition that comprises a radiopaque, unsaturated multi-arm polymer in accordance with the present disclosure that is reactive with the polyamino compound. The first and second compositions are generally sterile compositions, for example, sterilized by heat, sterile filtration, radiation or another suitable technique.

The first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise pH adjusting agents and/or additional agents such as those described below.

The second composition may be a second fluid composition comprising the radiopaque, unsaturated multi-arm polymer or a second dry composition that comprises the radiopaque, unsaturated multi-arm polymer, to which a suitable fluid such as water for injection, saline, etc. can be added to form a second fluid composition. In addition to the radiopaque, unsaturated multi-arm polymer, the second composition may further comprise pH adjusting agents and/or additional agents such as those described below.

In some embodiments, the polyamino compound is initially combined with the radiopaque, unsaturated multi-arm polymer at an acidic pH at which crosslinking between the unsaturated groups of the polymer and the amino groups of the polyamino compound is suppressed. Then, when crosslinking is desired, a pH of the mixture of the polyamino compound and the polymer is changed from an acidic pH to a basic pH, leading to crosslinking between same.

In particular embodiments, the system comprises (a) a first precursor composition that comprises a polyamino compound as described hereinabove, (b) a second precursor composition that comprises a radiopaque, unsaturated multi-arm polymer as described hereinabove, and (c) a third composition, specifically, an accelerant composition, that contains an accelerant that is configured to accelerate crosslinking reaction between the polyamino compound and the radiopaque, unsaturated multi-arm polymer.

The first precursor composition may be a first fluid composition comprising the polyamino compound that is buffered to an acidic pH or a first dry composition that comprises the polyamino compound and acidic buffering composition, to which a suitable fluid such as water for injection, saline, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 5, typically ranging from about 3.5 to about 4.5, and more typically ranging from about 3.8 to about 4.2. In addition to the polyamino compound and buffering species, the first precursor composition may further comprise additional agents, such as therapeutic agents and/or further imaging agents (beyond the iodine groups that are present in the polyamino compound).

The second precursor composition may be a second fluid composition comprising the radiopaque, unsaturated multi-arm polymer or a second dry composition that comprises the radiopaque, unsaturated multi-arm polymer from which a fluid composition is formed, for example, by the addition of a suitable fluid such as water for injection, saline, or by the addition of the first fluid composition comprising the polyamino compound that is buffered to an acidic pH. In addition to the radiopaque, unsaturated multi-arm polymer, the second precursor composition may further comprise additional agents, such as therapeutic agents and/or further imaging agents (beyond the iodine groups that are present in the radiopaque, unsaturated multi-arm polymer).

In a particularly beneficial embodiment, the first precursor composition is a first fluid composition comprising the polyamino compound that is buffered to an acidic pH and the second precursor composition comprises a dry composition that comprises the radiopaque, unsaturated multi-arm polymer. The first precursor composition may then be mixed with the second precursor composition thereby providing a prepared fluid composition that is buffered to an acidic pH and comprises both the polyamino compound and the radiopaque, unsaturated multi-arm polymer. In a particular example, a syringe may be provided that contains a first fluid composition comprising the polyamino compound that is buffered to an acidic pH, and a vial may be provided that comprises a dry composition (e.g., a powder) that comprises the radiopaque, unsaturated multi-arm polymer. The syringe may then be used to inject the first fluid composition into the vial containing the radiopaque, unsaturated multi-arm polymer to form a prepared fluid composition that contains the polyamino compound and the radiopaque, unsaturated multi-arm polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11, typically ranging from about 9.5 to about 10.5, and more typically ranging from about 9.8 to about 10.2. In addition to the above, the fluid accelerant composition may further comprise additional agents, such as therapeutic agents and/or further imaging agents (beyond the iodine groups that are present in the radiopaque, unsaturated multi-arm polymer). In a particular example, a syringe may be provided that contains a fluid accelerant composition.

A prepared fluid composition that is buffered to an acidic pH and comprises the polyamino compound and the radiopaque, unsaturated multi-arm polymer as described above, and a fluid accelerant composition that is buffered to basic pH, as described above, may be combined form crosslinked medical hydrogels, either in vivo or ex vivo.

Examples of further imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope ^{99m}Tc, as well as other gamma emitters such as ¹²³I, ¹²⁵I, ¹³¹I, ¹¹¹In, ⁵⁷Co, ¹⁵³Sm, ¹³³Xe, ⁵¹Cr, ^{81m}Kr, ²⁰¹Tl, ⁶⁷Ga, and ⁷⁵ Se, among others, (e) positron emitters, such as ¹⁸F, ¹¹C, ¹³N, ¹⁵O, and ⁶⁸Ga, among others, may be employed to yield functionalized radiotracer coatings, and (f) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters and drugs that may enhance neointimal formation such as the growth of endothelial cells, among others.

In various embodiments, a system is provided that include one or more delivery devices for delivering first and second compositions to a subject.

In some embodiments, the system may include a delivery device that comprises a first reservoir that contains a first composition that comprises a polyamino compound as described above and a second reservoir that contains a second composition that comprises a radiopaque, unsaturated multi-arm polymer as described above. In some embodiments, the system may include a delivery device that comprises a first reservoir that contains a first composition that comprises the polyamino compound and the radiopaque, unsaturated multi-arm polymer and is buffered to an acidic pH, such as the prepared fluid composition previously described, and a second reservoir that contains a second composition, such as the fluid accelerant composition described above. In either case, during operation, the first composition and the second composition are dispensed from the first and second reservoirs and combined, whereupon the polyamino compound and the radiopaque, unsaturated multi-arm polymer and crosslink with one another to form a hydrogel.

In particular embodiments, the system may include a delivery device that comprises a double-barrel syringe, which includes first barrel having a first barrel outlet, which first barrel contains the first composition, a first plunger that is movable in the first barrel, a second barrel having a second barrel outlet, which second barrel contains the second composition, and a second plunger that is movable in the second barrel.

Regardless of the first and second compositions selected, in some embodiments, the device may further comprise a mixing section having a first mixing section inlet in fluid communication with the first barrel outlet, a second mixing section inlet in fluid communication with the second barrel outlet, and a mixing section outlet. In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked medical hydrogel is ultimately formed at the administration location.

After administration, the compositions of the present disclosure can be imaged using a suitable imaging technique. Typically, the imaging techniques is an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked medical hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

In some aspects, the present disclosure pertains to medical kits that comprise (a) a first contain comprising a polyamino compound, (b) a second container comprising a radiopaque, unsaturated multi-arm polymer that comprises a plurality of hydrophilic polymer arms having end moieties comprising one or more radiopaque halogen groups and one or more unsaturated groups. The kit may further comprise one or more of the following: (a) a dual syringe injection device, (b) a catheter, (c) a needle, (d) one or more syringe barrels and (e) one or more fluids suitable for injection such as water for injection, saline, an acidic buffer and/or a basic buffer).

### EXAMPLE

A polyamino compound as described hereinabove is dissolved in acidic buffer solution (pH value between 3.8-4.2) and then further mixed with a radiopaque, unsaturated multi-arm polymer as described hereinabove to form a prepared fluid composition in one syringe. Another buffer solution is prepared having a controlled pH value around 9.8-10.4 as a fluid accelerant composition in another syringe. Subsequently, a hydrogel is formed by combining the prepared fluid composition and the fluid accelerant composition, for example, using a suitable double syringe-barrel injection device, to form a crosslinked medical hydrogel.

1. A system for forming a hydrogel that comprises (a) a polyamino compound and (b) a radiopaque, unsaturated multi-arm polymer that comprises a plurality of hydrophilic polymer arms having end moieties comprising one or more radiopaque halogen groups and one or more unsaturated groups that are reactive with amino groups of the polyamino compound, the end moieties linked to the hydrophilic polymer arms though an ester or an amide linkage.

2. The system of claim 1, wherein the polyamino compound comprises a plurality of —(CH₂)ₓ—NH₂ groups where x is 0, 1, 2 3, 4, 5 or 6.

3. The system of claim 2, wherein the plurality of —(CH₂)ₓ—NH₂ groups are disposed along a polymeric moiety.

4. The system of claim 1, herein the polyamino compound comprises two or more amino acid residues selected from residues of lysine, ornithine, and combinations thereof.

5. The system of claim 1, wherein the radiopaque halogen groups are iodine groups and wherein the one or more unsaturated groups are selected from acrylate ester groups and propiolate ester groups.

6. The system of claim 1, wherein the end moieties comprise a monocyclic or multicyclic aromatic moiety that is substituted with (a) one or more iodine groups and (b) one or more acrylate groups and/or one or more C₄-C₁₆-alkyl acrylate groups.

7. The system of claim 1, wherein the end moieties comprise a benzene ring that is substituted with one or more iodine groups and one or more acrylate groups or propiolate groups.

8. The system of claim 1, wherein the hydrophilic polymer arms are selected from polyether arms, poly(N-vinyl pyrrolidone) arms, polyoxazoline arms, poly(vinyl alcohol) arms, poly(allyl alcohol) arms, polyhydroxyethyl acrylate arms, polyhydroxyethyl methacrylate arms, PNIPAAM arms, and polysaccharide arms.

9. The system of claim 1, wherein the system comprises a first precursor composition that comprises the polyamino compound, a second precursor composition that comprises the radiopaque, unsaturated multi-arm polymer, and an optional accelerant composition.

10. The system of claim 1, wherein the system comprises a first precursor composition that comprises the polyamino compound in a first syringe barrel, a second precursor composition that comprises the radiopaque, unsaturated multi-arm polymer in a vial, and an accelerant composition in a second syringe barrel.

11. The system of claim 1, further comprising a delivery device.

12. A medical hydrogel formed by crosslinking the polyamino compound and the radiopaque, unsaturated multi-arm polymer of the system of claim 1 under conditions such that the medical hydrogel is formed.

13. The medical hydrogel of claim 12, wherein said medical hydrogel is a medical implant.

14. A method of treatment comprising administering to a subject a mixture that comprises a polyamino compound and a radiopaque, unsaturated multi-arm polymer that comprises a plurality of hydrophilic polymer arms having end moieties comprising one or more radiopaque halogen groups and one or more unsaturated groups that are reactive with amino groups of the polyamino compound under conditions such that the polyamino compound and the radiopaque, unsaturated multi-arm polymer cross-link after administration.

15. A radiopaque, unsaturated multi-arm polymer that comprises a plurality of hydrophilic polymer arms having end moieties comprising one or more radiopaque halogen groups and one or more unsaturated groups, the end moieties linked to the hydrophilic polymer arms though an ester or an amide linkage.

16. The radiopaque, unsaturated multi-arm polymer claim 15, wherein the hydrophilic polymer arms are selected from polyether arms, poly(N-vinyl pyrrolidone) arms, polyoxazoline arms, poly(vinyl alcohol) arms, poly(allyl alcohol) arms, polyhydroxyethyl acrylate arms, polyhydroxyethyl methacrylate arms, PNIPAAM arms, and polysaccharide arms.

17. The radiopaque, unsaturated multi-arm polymer of claim 15, wherein the hydrophilic polymer arms originate from a core region comprising a polyol residue or a core region comprising a silsesquioxane.

18. The radiopaque, unsaturated multi-arm polymer of claim 15, wherein the radiopaque halogen groups are iodine groups and wherein the one or more unsaturated groups are selected from acrylate ester groups and propiolate ester groups.

19. The radiopaque, unsaturated multi-arm polymer of claim 15, wherein the end moieties comprise a monocyclic or multicyclic aromatic moiety that is substituted with (a) one or more iodine groups and (b) one or more acrylate groups and/or one or more C₄-C₁₆-alkyl acrylate groups.

20. The radiopaque, unsaturated multi-arm polymer of claim 15, wherein the end moieties comprise a benzene ring that is substituted with one or more iodine groups and one or more acrylate groups or propiolate groups.
